(19) United States
(12) Patent Application Publication
(10) Pub. No.: US 2022/0151538 A1
(43) Pub. Date: May 19, 2022

(54) Oculomotor Testing Devices and Methods Using Add-On Structures for a Mobile Device

(71) Applicant: Bioeye Ltd., Hofit (IL)

(72) Inventors: Dovi YELLIN, Irus (IL); Marcus SCHWEIZER, Randwick (AU)

(21) Appl. No.: 17/591,320

(22) Filed: Feb. 2, 2022

Related U.S. Application Data
(63) Continuation-in-part of application No. 16/724,356, filed on Dec. 22, 2019.

(30) Foreign Application Priority Data
Jan. 31, 2022 (IL) 290265

(51) Int. Cl. A61B 5/398; A61B 5/00; A61B 3/00; A61B 3/113

(52) U.S. Cl. CPC A61B 5/398; A61B 5/0077; A61B 3/113; A61B 3/0083; A61B 5/6898

(57) ABSTRACT

A system for the administration of oculomotor tests with a mobile device is disclosed. The system includes: a cradle having a cavity configured to receive the mobile device therein; at least one light and at least one light sensor arranged on the cradle; an elongated light bar attachable to and electrically connectable to the cradle; and a stand including: an elongated frame configured to rest flat on a horizontal surface, the elongated frame comprising a first end, a second end, and a long axis defined between the first and second ends; a chin rest arranged at the first end; and a cradle support arranged at the second end and supporting the cradle thereon. The system facilitates the self-administration of a full stack of oculomotor tests.

### RELATED APPLICATIONS

This application is a continuation in part of U.S. patent application Ser. No. 16/724,356, filed Dec. 22, 2019, and claims priority to Israeli Patent Application No. 290,265, filed Jan. 31, 2022, entitled “Oculomotor Testing Devices and Methods Using Add-On Structures for a Mobile Device,” the contents of which are incorporated by reference as if fully set forth herein.

### FIELD OF THE INVENTION

This disclosure is generally directed to the field of monitoring neurophysiological performance. More specifically, this disclosure is directed to enhanced oculomotor testing devices and methods using an add-on structure to a mobile device.

### BACKGROUND OF THE INVENTION

Functions of the oculomotor nerve complex provide well-established methods for monitoring neurophysiologic performance. Known oculomotor tests for neurophysiological impairment detection include: the Near Point Convergence (NPC), the Horizontal Gaze Nystagmus (HGN), the Smooth Pursuit test, the Pupillary Light Reflex (PLR) checks and others. The validity of these tests for medical purposes has been substantiated in carefully devised scientific experiments, using highly accurate, complex and high-cost equipment. However, on a routine basis, when use of clinical gold-standard equipment is not attainable and commonplace state-of-art methods are in use, these tests are frequently not accurate enough, typically being administered manually by a human trained expert. In this scope, the human experts administering a test exert a subjective judgment, based on examinee performance, in order to provide a test score relative to some known norm, facilitating a diagnosis. Accordingly, taking advantage of recent technological development of mobile devices to overcome this subjective judgment, appears ideal. Yet, while wearable mobile devices are becoming more prevalent in the market-place, they have a variety of shortcomings in context of administering objective oculomotor tests, as detailed in the following section below.

International Patent Publication WO2021/113618, which is assigned to the same assignee as the present application, discloses a handheld add-on structure for a mobile device. The handheld add-on structure includes sensors and stimuli-providing lights and sensors suitable for conducting the tests described above. The mobile device coordinates performance of the tests using the components of the add-on device.

### SUMMARY OF THE INVENTION

Existing technical solutions for performance of oculomotor tests with mobile devices augmented by an add-on structure, including particularly the aforementioned patent publication, are directed to administration of the tests by a trained operator. Typically, the operator holds the add-on structure, with a screen of the mobile device facing the operator, and the back side of the mobile device facing the subject. While this configuration does result in advantages in administration of the test compared to tests administered without the benefit of such mobile devices and add-on structure, there are still limitations of such systems. First, since the add-on structure is itself handheld, it is necessary for the operator to be present and holding the device during the administration of the test. This feature thus still requires the active involvement of the operator in the test, and subjects the administration of the test to the potential for error by the operator. Second, because the add-on structure is handheld, it is necessary to include additional features in the add-on structure, such as a gimbal system, to ensure that the structure remains stable during administration of the test. These stabilizing structures add to the overall cost and complexity of the system.

Another technical solution for the performance of certain oculomotor tests involves the performance of the tests using virtual reality (VR) or augmented reality (AR) goggles. However, a disadvantage of performance of the tests using such devices is that the goggles at least partially (in the case of AR) or totally (in the case of VR) obscure the view of the subject's eyes during performance of the tests. In addition, the goggles may be heavy uncomfortable for the subject to wear, which, in turn, may influence the subject's ability to focus during performance of the tests.

Accordingly, it is an objective of the present disclosure to devise an add-on structure for a mobile device, suitable for administration of oculomotor tests by the patient, without active involvement of the operator. It is a further objective of the present disclosure to devise an add-on structure that is intrinsically stable without requiring the use of gimbals or similar balancing apparatuses.

According to a first aspect, a system for the administration of oculomotor tests with a mobile device is disclosed. The system includes: a cradle having a cavity configured to receive the mobile device therein; at least one light and at least one light sensor arranged on the cradle; an elongated light bar attachable to and electrically connectable to the cradle; and a stand including: an elongated frame configured to rest flat on a horizontal surface, the elongated frame comprising a first end, a second end, and a long axis defined between the first and second ends; a chin rest arranged at the first end; and a cradle support arranged at the second end and supporting the cradle thereon.

In another implementation according to the first aspect, the chin rest is foldable between an upright position and a folded position, wherein in the upright position the chin rest is perpendicular to the long axis, and in the folded position the chin rest is parallel to the long axis. The cradle support is foldable between an upright position and a folded position, wherein in the upright position the cradle support is perpendicular to the long axis, and in the folded position the cradle support is parallel to the long axis. When the chin rest and cradle support are both in the upright position, the chin rest and cradle support are parallel to each other.

In another implementation according to the first aspect, the length of the long axis is approximately 40 cm.

In another implementation according to the first aspect, the cradle support and cradle are removably attached to each other.

In another implementation according to the first aspect, the system further includes an adapter formed of a thermoplastic polymer, wherein said adapter is configured to be arranged within the cavity between the mobile device and the cradle to thereby stabilize the mobile device.

In another implementation according to the first aspect, the at least one light comprises a white light source configured to provide stimuli for relevant oculomotor tests and an infrared light source arranged above the cavity and configured to provide infrared light for purposes of improving facial IR imaging.

In another implementation according to the first aspect, the at least one light sensor comprises an infrared sensor arranged below the cavity.

In another implementation according to the first aspect, the light bar is attachable to the cradle in a first orientation in which the light bar is attached to the cradle at a centerpoint of the light bar, and is centered over the cradle, and a second orientation in which the light bar is attached to the cradle at an edge of the light bar, and overhangs over the elongated frame.

In another implementation according to the first aspect, the system further includes a connector arranged within the cradle for forming a data connection between the cradle and the mobile device.

In another implementation according to the first aspect, the system further includes an application stored in a non-transitory memory of the mobile device, said application configured to coordinate administration of one or more oculomotor tests using one or more of the at least one light, at least one light sensor, and the light bar. Optionally, the application is further configured to coordinate administration of the one or more oculomotor tests using a built-in sensor of the mobile device. Optionally, the application includes instructions for self-administration of the one or more oculomotor tests for a subject whose chin is resting on the chin rest.

According to a second aspect, a method is disclosed. The method includes: inserting a mobile device into a cavity of a cradle, the cradle being attached to a cradle support of a stand, wherein the stand comprises an elongated frame configured to rest flat on a horizontal surface, the elongated frame comprising a first end, a second end, and a long axis defined between the first and second ends; a chin rest arranged at the first end; and the cradle support arranged at the second end, and the cradle comprises the cavity configured to receive a mobile device therein, at least one light, and at least one light sensor arranged on the cradle; forming a data connection between the mobile device and the cradle; and operating an application stored in a non-transitory memory of the mobile device, thereby coordinating administration of one or more oculomotor tests, on a subject whose chin is resting on the chin rest, using one or more of the at least one light and the at least one light sensor.

In another implementation according to the second aspect, the one or more oculomotor tests includes a pupillary light reflex test or a smooth pursuit test.

In another implementation according to the second aspect, the method further includes attaching a light bar to the cradle, and the operating step comprises coordinating administration of the one or more oculomotor tests using the light bar.

In another implementation according to the second aspect, the step of attaching the light bar comprises attaching the light bar to the cradle at a centerpoint of the light bar, such that the light bar is centered over the cradle, and the one or more oculomotor tests comprises a horizontal gaze nystagmus test.

In another implementation according to the second aspect, the step of attaching the light bar comprises attaching the light bar to the cradle at an edge of the light bar, such that the light bar overhangs over the elongated frame, and the one or more oculomotor tests comprises a near point convergence test.

In another implementation according to the second aspect, the operating step includes coordinating administration of the one or more oculomotor tests using a built-in sensor of the mobile device.

In another implementation according to the second aspect, the operating step includes self-administering the one or more oculomotor tests.

In another implementation according to the second aspect, the method further includes opening the stand from a folded position, in which the chin rest and cradle support are parallel to the long axis, to an upright position, in which the chin rest and cradle support are in an upright position, perpendicular to the long axis, and parallel to each other.

In another implementation according to the second aspect, the method further includes, prior to inserting the mobile device into the cavity, attaching the cradle to the cradle support.

In another implementation according to the second aspect, the method further includes arranging an adapter made of a thermoplastic polymer within the cavity between the mobile device and the cradle, to thereby stabilize the mobile device.

According to a third aspect, a system for the administration of oculomotor tests with a mobile device is disclosed. The system includes a cradle having a cavity configured to receive the mobile device therein and an infrared light source configured to provide infrared light for purposes of facial infrared image sensing. A connector is arranged within the cradle for forming a power connection between the cradle and the mobile device, serving to power the infrared light source. A filter is arranged on an image sensor of the mobile device and is configured to pass infrared light and to block visible light.

In another implementation according to the third aspect, the connector further provides a data connection between the cradle and the mobile device, and the cradle further includes a white light source configured to provide stimuli for relevant oculomotor tests.

In another implementation according to the third aspect, the connector further provides a data connection between the cradle and the mobile device, and the system further includes an elongated light bar attachable to and electrically connectable to the cradle.

### BRIEF DESCRIPTION OF THE DRAWINGS

For a more complete understanding of this disclosure and its features, reference is now made to the following description, taken in conjunction with the accompanying drawings, in which:

FIG. 1 depicts a perspective view of a first embodiment of an add-on structure for a mobile device, with a light bar oriented parallel to a long axis of the add-on structure, according to embodiments of the present disclosure;

FIG. 2 depicts the add-on structure of FIG. 1 with the light bar oriented perpendicular to the long axis of the add-on structure, according to embodiments of the present disclosure;

FIGS. 3A and 3B depict exploded views of the embodiment of FIG. 1, according to embodiments of the present disclosure;

FIG. 4A illustrates a pattern of lights being displayed on the light bar during administration of the Horizontal Gaze Nystagmus test, according to embodiments of the present disclosure;

FIG. 4B illustrates a pattern of lights being displayed on the light bar during administration of the Near Point Convergence test, according to embodiments of the present disclosure;

FIG. 5A illustrates a patient self-administering the Horizontal Gaze Nystagmus (HGN) test using the add-on structure in the configuration of FIG. 4A, according to embodiments of the present disclosure;

FIG. 5B illustrates a patient self-administering the Near Point Convergence test using the add-on structure in the configuration of FIG. 4B, according to embodiments of the present disclosure;

FIG. 6 illustrates a process of inserting a mobile device into a cradle of the add-on device, according to embodiments of the present disclosure;

FIGS. 7A-7D illustrate a process of folding a stand of the add-on device of FIG. 1 into a folded position, according to embodiments of the present disclosure;

FIG. 8A discloses a back side of a second embodiment of an integrated device including a mobile phone and an add-on structure; according to embodiments of the present disclosure;

FIG. 8B discloses a front side of the integrated device of FIG. 8A, according to embodiments of the present disclosure;

FIG. 9 shows the location of the stimuli and how the screen can be moved toward a subject for a nearing or retracting dot scenario test (as in a NPC test), according to embodiments of the present disclosure;

FIG. 10 shows an example use of the device of FIGS. 8A, 8B, and 9 with a subject, according to embodiments of the disclosure;

FIGS. 11A, 11B, and 11C illustrate varying holders and corresponding features that may be utilized with the embodiment of FIGS. 8A, 8B, and 9, according to embodiments of the disclosure;

FIG. 12 illustrates another aspect of capturing distance of a subject with reference to the mobile device, according to embodiments of the present disclosure;

FIGS. 13A and 13B illustrate additional measurement techniques, according to embodiments of the disclosure;

FIG. 14 shows a simplified block diagram illustrative of a communication system that can be utilized to facilitate communication between endpoint(s) through a communication network, according to particular embodiments of the disclosure;

FIG. 15 is an embodiment of a general-purpose computer that may be used in connection with other embodiments of the disclosure to carry out any of the above-referenced functions and/or serve as a mobile computing device for endpoint(s); and

FIGS. 16A and 16B show a third configuration of an integrated device including a mobile phone and an add-on structure, according to embodiments of the present disclosure.

### DETAILED DESCRIPTION OF THE INVENTION

The FIGURES described herein, and the various embodiments used to describe the principles of the present disclosure in this patent document are by way of illustration only and should not be construed in any way to limit the scope of the disclosure. Those skilled in the art will understand that the principles of the present disclosure may be implemented in any type of suitably arranged device or system. Additionally, the drawings are not necessarily drawn to scale.

As indicated earlier, functions of the oculomotor nerve complex provide well-established methods for monitoring neurophysiologic performance. Known oculomotor tests for neurophysiological impairment detection include: the Near Point Convergence (NPC), the Horizontal Gaze Nystagmus (HGN), the Smooth Pursuit test, the Pupillary Light Reflex (PLR) checks and others. The validity of these tests for medical purposes has been substantiated in carefully devised scientific experiments, using highly accurate, complex and high-cost equipment. However, on a routine basis, when use of clinical gold-standard equipment is not attainable and commonplace state-of-art methods are in use, these tests are frequently not accurate enough, typically being administered manually by a human expert. Human experts exert a subjective judgment, based on examinee performance, in order to provide a test score relative to some known norm, facilitating a diagnosis.

Mobile devices are becoming more and more powerful processing tools; however, by themselves, they are still insufficient for reliable oculomotor tests. As a non-limiting example, even mobile devices with a high definition camera (FHD-1920×1080 pixels or even UHD-3840×2160 pixels resolution) have insufficient ability to capture eye-movement in low light conditions. When such mobile devices attempt to add light (e.g., from a flash) to rectify the low-light conditions, the prolonged light distorts the testing. Light reflection and glints add an additional source of noise to images obtained by normal light cameras. In the alternative, while certain mobile devices may have infrared features, such infrared features are not designed for the image context of eye-marker extraction and are thus unsuitable for oculomotor tests.

Given these difficulties, embodiments of the disclosure provide testing using a standard mobile device, such as a smartphone, supplemented as necessary with an add-on structure that has a two-way communication with the mobile device. When combined with an application installed on the mobile device, the combined device enables accurate and consistent administration of oculomotor functional tests. In particular, the combined device in specific embodiments enables the detection of neurophysiological impairment measured by, for example, visual pursuit tracking, pupillometry, vergence, eye-lid tremor, blink metrics (such as the mean and variance of blink rate, duration, closure speed etc.) and gaze nystagmus tests. In certain embodiments, the combined device may be used to automate and facilitate these oculomotor tests to detect impairment due to consumption of toxic substances (such as drugs and alcohol), as well as the monitoring of impairment due to mild traumatic brain injuries (as in sports concussions) and sleep deprivation. While certain impairments will be described herein, it should be understood that such are intended as being non-limiting examples. The same disclosure herein may also be used for other types of impairments—including those associated with after-arising technologies and techniques and non-neurological related impairments (e.g., other medical or biological related impairments). Also, while certain tests are referenced, other tests may be performed.

The following literature provides examples of oculomotor tests and feedback for determining whether a condition exists:
1. Kulkarni et al (2015). Ocular manifestations of head injury: a clinical study. Eye 19(12):1257-63
2. Miller (2006) Neuro-ophthalmologic manifestations of psychogenic disease. Semin Neurol. 26(3):310-20
3. Patel and Lundy (2002) Ocular manifestations of autoimmune disease. Am. Fam. Physician. 15; 66(6):991-8
4. Cohen et al (1989) Convergence Insufficiency in Brain Injured Patients. Brain Inj: 3: 187-191.
5. Bruke et al (1993) Convergence Insufficiency in Thyroid Eye Disease. J. Pediatr. Ophthalmol. Strabismus. 30: 127-129.
6. Biousse et al. (2004) Ophthalmologic features of Parkinson's Disease. Neurology 62: 177-180.
7. Ciuffreda et al (2017). Understanding the effects of mild traumatic brain injury on the pupillary light reflex. Concussion. 2(3).
8. Garner et al (2018). Blink reflex parameters in baseline, active, and head-impact Division I athletes. Cogent Engineering 5(1), 1429110.
9. Serra and Leigh (2002) Diagnostic value of nystagmus: spontaneous and induced ocular oscillations. Journal of Neurology, Neuros. & Psych. 73:615-618.
10. Poynter (2017). Pupil-size asymmetry is a physiologic trait related to gender, attentional function, and personality. Laterality. 22(6):654-670.
   Again, while such example tests are described, other tests—including those that have yet to be developed—may avail from the disclosures herein.

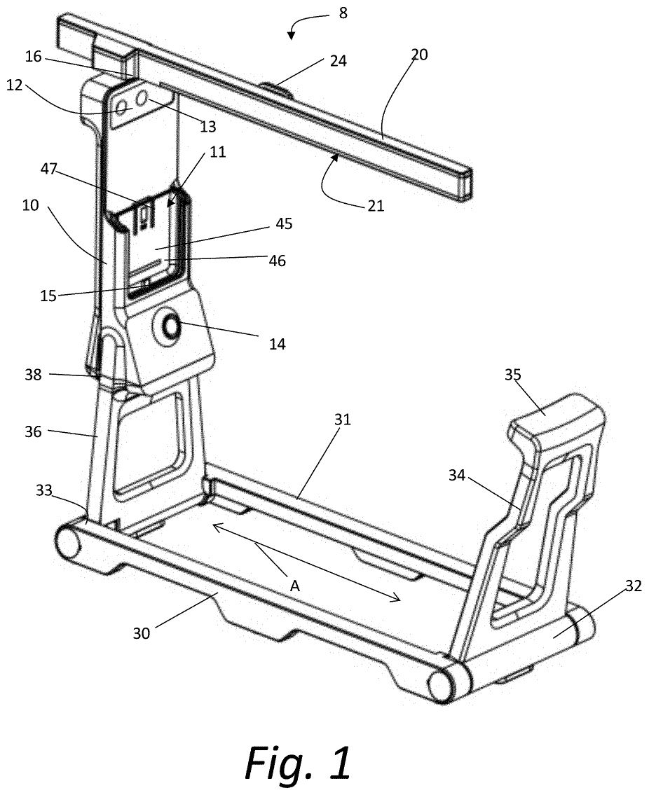

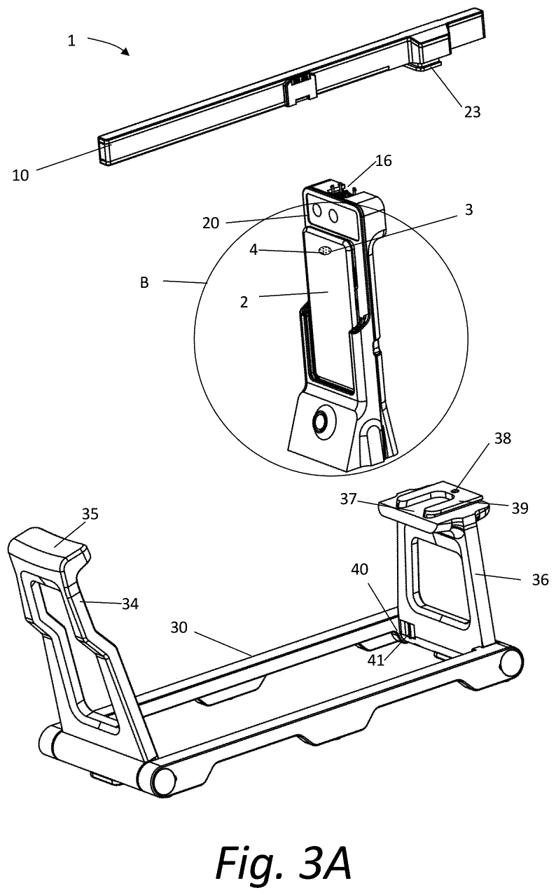

Referring now to FIGS. 1-7, a first embodiment of a system 1 for the administration of oculomotor tests with a handheld stabilized mobile device 2 is disclosed. System 1 includes an add-on device 8 comprised of cradle 10, an elongated light bar 20, and a stand 30.

Cradle 10 includes a cavity 11 that is generally shaped to enclose and support a mobile device 2, such as a smartphone or a tablet. In the illustrated embodiment, the mobile device 2 is a smartphone; however, this is merely exemplary, and the mobile device 2, and correspondingly the cavity 11, may be sized for a tablet computer as well.

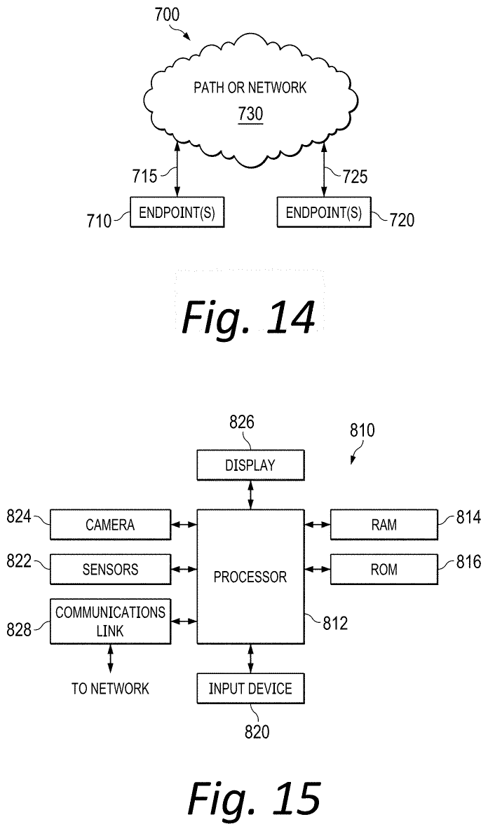

The mobile device 2 may be a commercially available smartphone—including those currently available from manufacturers such as Apple (e.g., the iPhone), Google (e.g., Pixel phones), Samsung (e.g., Galaxy Phones), and others. In other configurations, the mobile device 2 may comprise other commercially available devices such as tablet computers or smart watches. A general description of capabilities of the mobile device 2 is described with reference to FIG. 15, including features such as processors, cameras, operating systems, RAM, communication capability, and the like.

In order to accommodate for differences in sizes of various mobile devices 2, the cradle 10 may include an adapter 45. The adapter 45 may be made of a thermoplastic polymer, for example a blend of acrylonitrile butadiene styrene (ABS) and thermoplastic polyurethane (TPU). Adapter 45 may include a TPU ring 46 arranged on an inner circumference thereof for forming an interference fit with the mobile device 2. Adapter 45 may further include a rear clip 47 which is configured to form a snap-fit connection with a corresponding groove 48 in the cradle 10. The adapter 45 further includes a hole 49 at the center of a bottom portion thereof, suitable for the fixation (or insertion) of a power connector (such as a USB-C connector) therethrough. Adapter 45 may be sized in order to match the dimensions of any suitable mobile device 2.

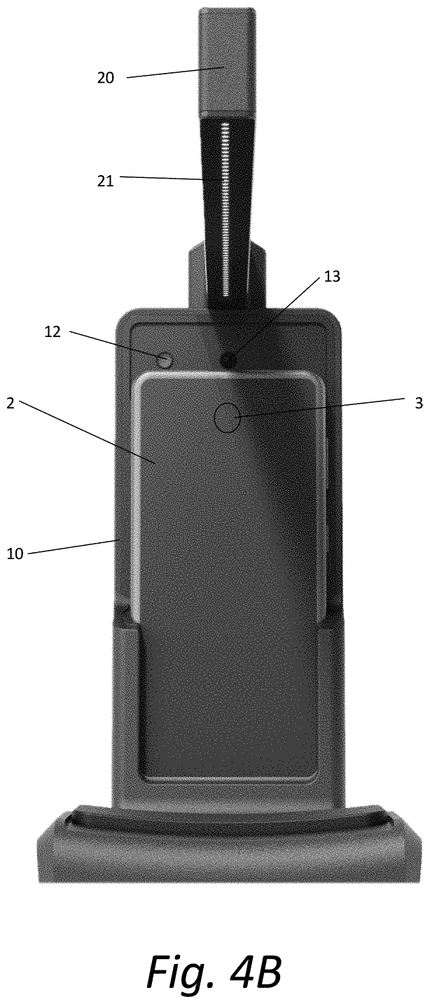

During an attachment of the mobile device 2 to the cradle 10, the mobile device 2 may first be inserted into the adapter 45. The combined mobile device 2 and adapter 45 may be guided into the cavity 11, until the rear clip 47 snaps into the groove 48. This process is shown in FIG. 6. Alternatively, the adapter 45 may first be inserted and snapped into the cavity 11, and then the mobile device 2 is inserted into the adapter 45.

Cradle 10 includes a connector 15. Connector 15 is any suitable connector for power and/or data connection, such as a USB-C connector. The connector 15 is fixated within the cavity such that it is connectable with a corresponding receptacle in the mobile device 2, to thereby form a power and data connection. In such configurations, the cradle 10 may receive power from the mobile device 2 and may share data back and forth with mobile device 2 via connector 15. A data connection may alternatively be achieved without the use of connector 15, such as through Bluetooth, WiFi, or another wireless form of data transfer. Similarly, in alternative embodiments, cradle 10 may include a separate power source in addition or instead of connector 15, such as a rechargeable battery.

Cradle 10 further includes at least one light source, for use in performance of oculomotor testing. In exemplary embodiments, the at least one light source includes a source of white light 12 (for providing the PLR-related stimulus) and a source of infrared light 13 (facilitating infrared-based capture of the subject's eyes). In the illustrated embodiments, light sources 12, 13 are arranged on the cradle 10 above the cavity 11.

White light source 12 is, in exemplary embodiments, a bright light-emitting diode. The white light source 12 may be designed for optimized pupillary light reflex (PLR)-related light flash, so that the pupil responds clearly to the stimuli, but without causing any long-term damaging effect to the participant's eyes. In particular configurations, a 450 nm wavelength light may be used for approximately one second. In other configurations, different wavelengths and different timing may be used.

Infrared light source 13 is configured to illuminate a subject's eyes with infrared light that is reflected off of the subject's eyes, as is appropriate for relevant tests. In addition to, or instead of, infrared light source 13 being located on a top portion of the cradle 10, one or more sources of infrared light may be located on the light bar 20.

Infrared sensor 14 is configured below the cavity 11 and is used to sense infrared light reflected off of a subject. IR sensor 14 may be high-definition, having a resolution of at least 1920 by 1080 pixels. In other configurations, the resolution may be even higher. Optimal sensitivity of the infrared sensor 14, in a typical embodiment, is at wavelength of 850 nm. While a single infrared sensor 14 is shown in this configuration, in other configurations multiple infrared sensors may be utilized.

In alternative embodiments, the residual reflected infrared light is sensed using a forward-facing camera or image sensor 3 of the mobile device 2. Typically, a camera 3 of a mobile device includes a CMOS sensor that is sensitive to both visible and infrared light. To use the camera 3 of mobile device 2 as an infrared sensor, a filter 4 (shown as a pattern within camera 3 in some Figures) may be applied to the camera 3, blocking wavelengths in the visible range and permitting passage only of near infrared wavelengths.

The use of infrared sensor 14 and infrared light source 13 enables overcoming normal smartphone camera limitations relating to: dynamic lighting conditions (brightness, backlight, reflections), dark eyes (lack of contrast for some users), dynamic background, and the like. Providing such features allows the system 1 to benefit from capturing the image with an IR camera of high resolution in order to apply both full face and eyes image processing techniques, as well as known IR-oriented pupil extraction methods.

In still other embodiments, the image sensor 3 of the mobile device 2 may be used in conjunction with the infrared sensor 14. For example, the information captured by the IR sensor 14 may be utilized for detecting eye movement, while the image sensor 3 of the mobile device 2 may be used for alignment. In other configurations, the eye movement may be detected by a combination of the IR sensor 14 and the image sensor 3 of the mobile device 2.

Furthermore, in certain embodiments, the cradle 10 may lack any image sensor, and the system may instead rely entirely on the image sensor 3 of the mobile device 2, filtered as appropriate with filter 4, for capturing of infrared light. In such embodiments, the cradle may include the cavity and the infrared light source configured to provide infrared light for purposes of improving facial infrared image sensing. In basic embodiments with this configuration, the connector 15 provides only a power connection between the mobile device 2 and the cradle 10, for powering the infrared light source. The mobile device 2 may be used in with such a cradle 10 for eye tracking. This basic embodiment thus requires fewer components, and correspondingly less expense, than system described above. In the alternative, the connector 15 may also provide a data connection, such that the cradle 10 may also provide stimuli for oculomotor testing. In such embodiments, the cradle may still have a white light for providing stimuli for conducting of relevant oculomotor tests, and the system may further include a light bar for providing additional stimuli, as set forth herein.

Cradle 10 further includes a port 16 at an upper portion thereof. The port 16 includes pins or other suitable connectors for receiving, and forming an electrical connection, with the elongated light bar 20.

Cradle 10 further includes a printed circuit board (not shown) arranged within the cradle 10. The printed circuit board includes a processor and a memory. The memory includes computer program instructions, that, when executed by the memory, cause the various electrical components described herein to perform the oculomotor tests described herein. The processor is further configured to receive instructions from an application running on the mobile device 2, and to transmit sensor readings to the mobile device 2.

Other sensors may optionally be utilized and incorporated in the cradle 10. For example, Time-of-Flight (ToF) cameras may be utilized. LIDAR systems (including associated sensors) may also be utilized. Yet other sensors may alternatively be utilized while remaining in the scope of this disclosure, including but not limited to, those associated with photogrammetry.

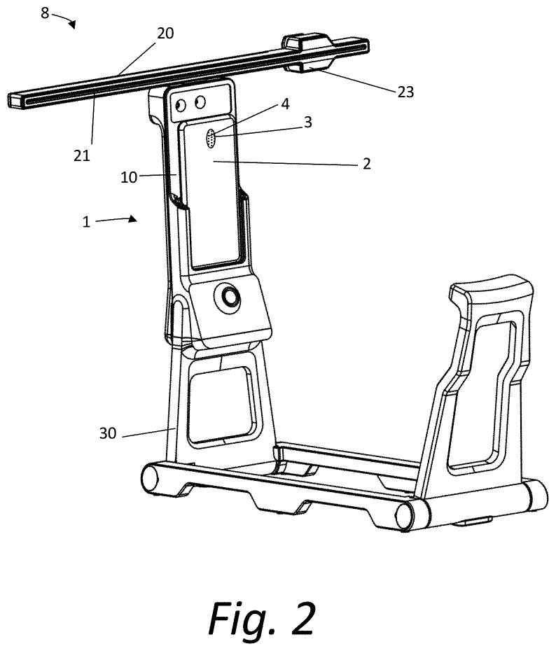

Elongated light bar 20 includes a line of LED lights 21 that extends along the long axis of the light bar 20. The LED lights 21 may be turned on and off individually, in a series, as will be described further herein. Light bar 20 further includes two connector ports 23, 24 for connection with port 16 of the cradle in a first orientation and a second orientation. Port 23 is on the same face of the light bar 20 as the LED lights 21, and is configured at an edge of the light bar 20. Connector port 23 is used when the add-on device 1 is in the orientation as shown in FIGS. 1, 4B, and 5B, and results in the light bar 20 overhanging over stand 30, for performance of the Near Point Convergence test. Connector port 24 is oriented at a 90 degree angle to the LED lights 21, and is configured at the center of the light bar 20. Connector port 24 is used when the add-on device 1 is in the orientation of FIGS. 2, 4A, and 5A, and results in the light bar 20 being centered with respect to cradle 10, for performance of the Horizontal Gaze Nystagmus test.

The length of the light bar 20 is approximately 40 cm. This length facilitates approximately a 30 degree angle of view at the edge points of the light bar 20 during performance of the horizontal gaze nystagmus test. This angle presumes a distance of approximately 40 cm between the light bar and the subject's eyes, and the stimuli at the outer edges of the light bar 20 being 20 cm from the center of the light bar 20. The width of the face of the light bar 20 containing LED lights 21 may be approximately 1 cm. Such a width may facilitate a good view of a dot moving from edge to edge of the light bar 20 at a preconfigured pace. The diameter of each dot may be approximately 0.5 cm. The color of the LED lights may be any suitable color, such as green or white.

Stand 30 is comprised of three elements: an elongated frame 31, a chin rest 32, and a cradle support 36. Frame 31 includes support members arranged in a generally rectangular fashion, extending between first end 32 and second end 33. The support members may be made of any suitable material and shape to ensure that the stand 30 rests stably on a flat surface. The support members define a long axis “A” between the first end 32 and second end 33. In exemplary embodiments, the length of the long axis is approximately 40 cm. This length results in proper positioning of the subject's eyes for performing the tests described herein.

Chin rest 34 is arranged at the first end and includes chin surface 35, on which a subject rests his or her head during performance of the oculomotor tests.

Cradle support 36 is arranged at the second end 33 and includes a surface 37 on which the cradle 10 rests during performance of the oculomotor tests. Cradle support 36 further includes an attachment mechanism 38 for securely connecting the cradle 10 to the cradle support. The attachment mechanism 38 may include, for example, aligned bores through which a bolt (not shown) may be threaded. In addition, cradle support 30 may have a protruding ridge 39, which may correspond to an indentation (not shown) in the bottom of the cradle 10. The aligned ridge 39 and indentation help secure the cradle 10 from lateral displacement.

The heights of the chin rest 34 and cradle support 36 are set for comfort of the user, as well for proper placement of the user's eyes relative to the stimuli generated, and the need to capture images of the eyes accurately, by the add-on device 8.

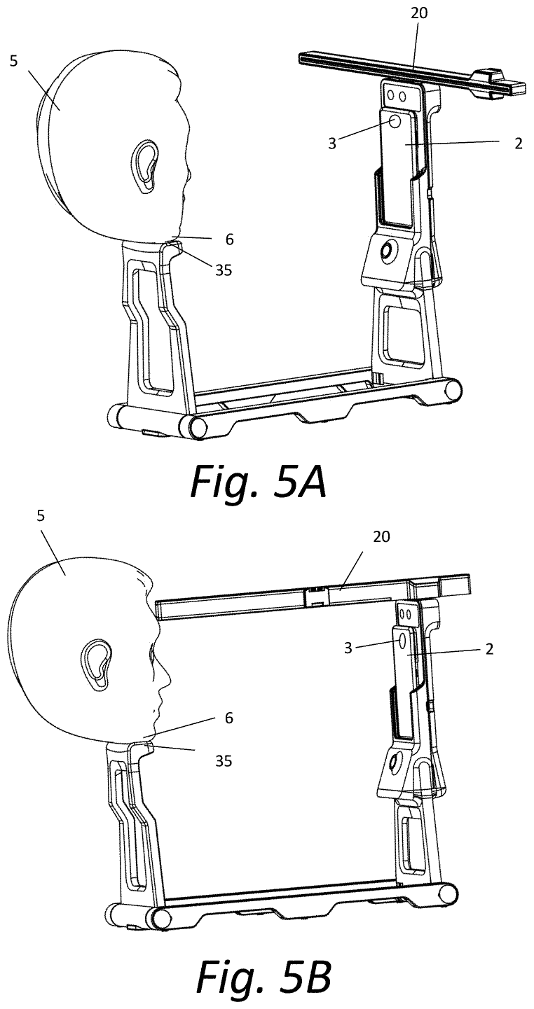

In preferred embodiments, and as shown in FIGS. 7A-7D, both the chin rest 34 and cradle support 36 are foldable between upright and folded positions. In the upright position, the chin rest 34 is perpendicular to the long axis A, as shown in FIGS. 1-6; and in the folded position, the chin rest 34 is parallel to the long axis A. Similarly, the cradle support 36 may be foldable between an upright position and a folded position. In the upright position, the cradle support 36 is perpendicular to long axis A, as shown in FIGS. 1-6, and in the folded position the cradle support is parallel to the long axis A. FIGS. 7A-7D show a process of folding the stand 30. In FIG. 7A, cradle 10 is removed from stand 30, leaving the stand 30 as shown in FIG. 7B. In FIG. 7C, the chin rest 34 is folded clockwise toward the center of the frame 31, and in FIG. 7D, the cradle support is folded counterclockwise, also toward the center of frame 31. In alternative embodiments, it is possible to fold the chin rest 34 and/or the cradle support 36 in the opposite directions.

Each of the chin rest 34 and cradle support 36 may include a torsion spring (not shown) at or adjacent to the first end 32 or the second end 33, the torsion spring being configured to bias the chin rest 34 and cradle support 36 to the folded positions. Similarly, the chin rest 34 and cradle support 36 may include one or more locking tabs 41 that may hold the chin rest 34 and cradle 36 in place against the force of the respective springs, in the manner known to those of skill in the art.

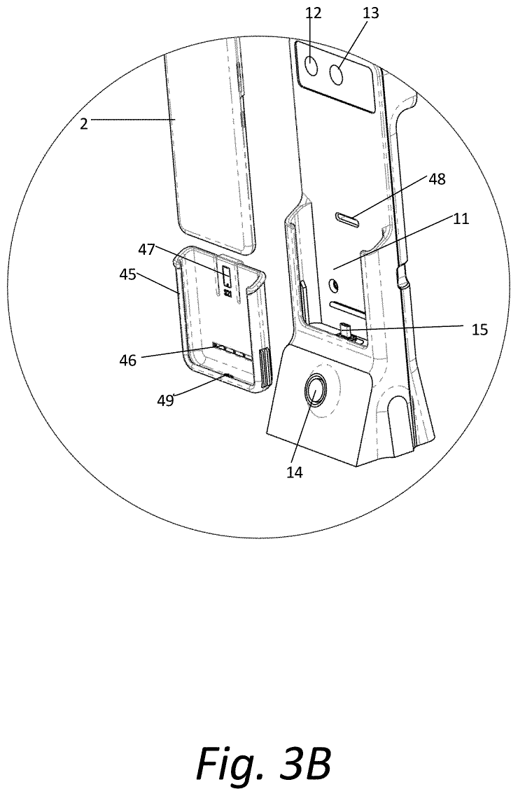

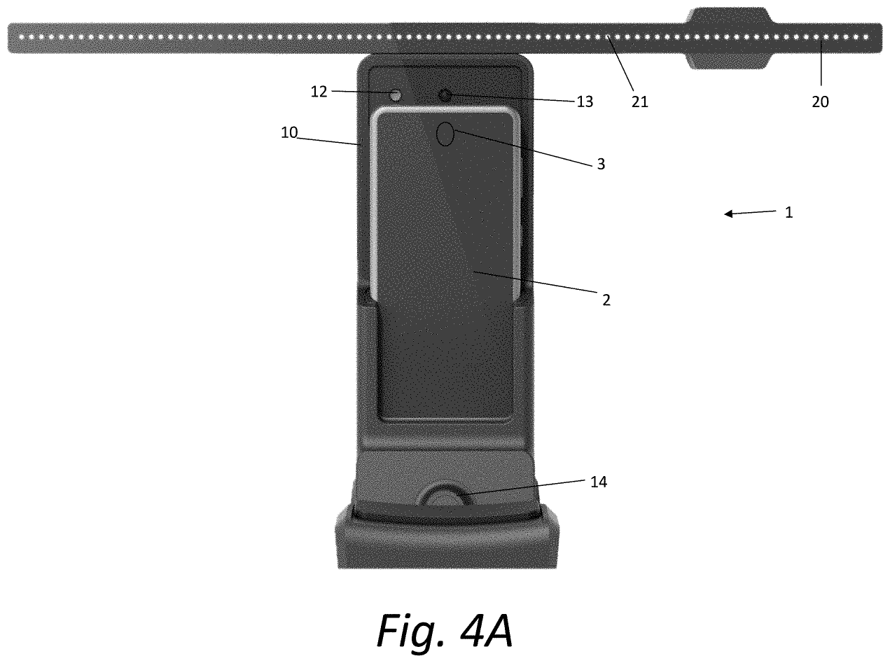

Referring now to FIGS. 4A, 4B, 5A, and 5B, the mobile device 2 includes an application stored in a non-transitory memory of the mobile device 2, which is configured to coordinate administration of the oculomotor tests. The application may further deliver instructions to the user regarding how to administer the tests to another subject, or how to self-administer the tests. For example, the application may display instructions on a screen of the mobile device or play instructions through a speaker of the mobile device.

The application installed on mobile device 2 may correspond to an account with which information may be stored and recalled (e.g., on a different mobile device 2) by simply logging into (or authenticating as appropriate) an account. Any suitable authentication protocol may be used for such embodiments.

In particular configurations, the application may provide instructions to a user or administrator regarding what needs to be done next. Or, if appropriate readings were not obtained in a given test, the application may provide instructions to repeat a test.

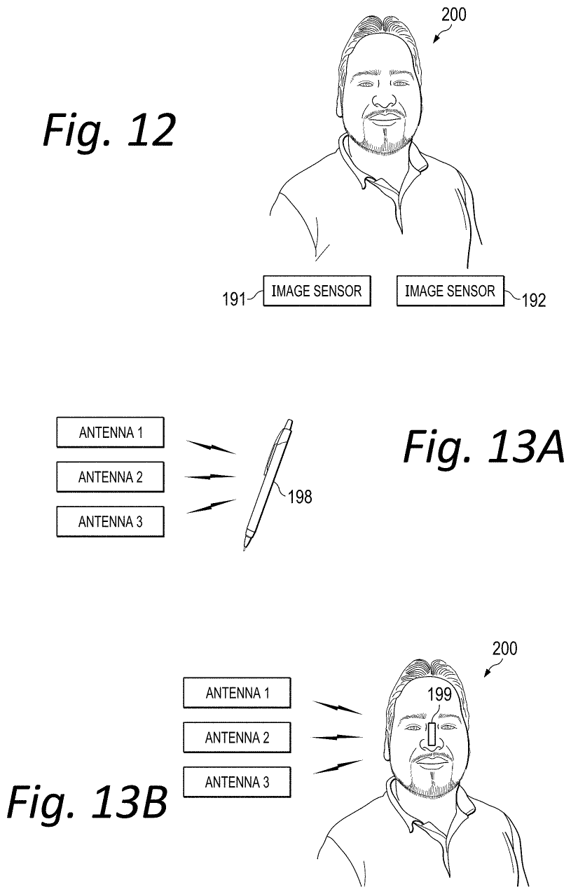

In general operation, a two-way communication exists between the add-on structure 1 and the mobile device 2—passing instruction and information back and forth to conduct the oculomotor tests. As an example, instructions on timing and pace of stimulation may be provided by the mobile device 2 to add-on structure 1 on the one hand, and IR sensor information may be provided from the latter back to the former. Image information from the infrared sensor 14 is served into the mobile device 2 for algorithmic image processing and inference of eye-markers using advanced machine learning solutions. One example of such solutions is disclosed in International Patent Publication WO2018/142388A1, which is assigned to the same assignee as the present application, and a copy of which is incorporated by reference as if fully set forth herein. The processing may include other approaches, such as the use of state-of-the-art convolutional or visual transformer deep learning methods. While processing of these algorithms for eye-marker extraction may be performed locally on the mobile device 2, in other configurations, the mobile device 2 may off-load some, none, or all of the processing to a remote device (e.g., servers), using communication capabilities of the mobile device 2. Non-limiting example of communication are described with reference to FIG. 14.

In particular configurations, portions of tests, or all of tests, may be conducted in an automated fashion. In particular, a subject or administrator, upon selecting within the application a particular test to be performed, may initiate the testing with the mobile device 2 in an automated, preconfigured fashion. Little or no human intervention may be required for a test at all. In such automated testing, using dynamic feedback received from measurement of the eyes, stimuli lights from light bar 20 may start moving after a participant eye calibration phase is performed. When a certain movement of the eye needs to be reviewed more carefully, the application may change the speed and movement of the moving dot representation. In the automated operations, the processing for dynamic feedback to change the test may be performed locally, remotely, or a combination of both.

During administration of the test, a subject 5 places his or her chin 6 on the chin surface 35, as shown in FIGS. 5A and 5B. The patient gazes at the cradle 10 and light bar 20, and follows instructions that are communicated by the mobile device 2 (e.g., on the screen of the mobile device 2 and/or via a speaker built in to the mobile device 2).

Although a particular advantage of the embodiments of FIGS. 1-7 is their ability to be utilized for self-administration, it is also possible for an administrator to exert greater control over the performance of the tests. For example, the administrator may control operation of the application through a separate device that is connected wirelessly to the mobile device 2. In one embodiment, the administrator may move the stimuli dots on the light bar 20 using a virtual touch-screen slider on a separate device. Such embodiments may be advantageous for administration of tests by seasoned Drug Recognition Evaluators (DRE), for example, who may have a desire to have more control over the testing processes.

The following are non-limiting examples of oculomotor tests that may be conducted by the system 1, and the other embodiments disclosed herein, according to embodiments of the disclosure. While such tests are examples, the integrated devices herein (including system 1) may be utilized for other tests—including after-arising tests that are later developed.

Horizontal Gaze Nystagmus (HGN) is a biphasic ocular oscillation alternating a slow horizontal eye movement, or smooth pursuit, in one direction and a fast jerky eye movement, or saccadic movement, in the other direction. The velocity of the slow phase eye movement (SPEV) and the fast phase eyes velocity (FPEV) are related to each other and can be considered as a measurement of the efficiency of the system stimulus/response. Particular embodiments may also use vertical gaze nystagmus (VGN) testing.

When the horizontal gaze nystagmus test is performed, as shown in FIGS. 4A and 5A, a series of lights 21 may be shown starting in the center of the light bar 20, and proceeding to one or the other edge of the light bar 20, providing a stimulus that appears as a moving dot, to be tracked by the subject. The image sensor 14 captures the eyes as they track the lights, and uses image processing and machine learning algorithms to evaluate whether there is any involuntary lateral or horizontal jerking movement. In exemplary embodiments, a complete cycle for an automated horizontal gaze nystagmus test (e.g., one full movement of the dot from center all the way to left, back to center, then all the way to right and finally back to center) may be completed in approximately 10 seconds.

Near Point Convergence (NPC) provides a measure of pursuit convergence by testing closer and closer points of gaze on an object (in common practice a finger or pencil moved by the examining physician) held in front of the eyes, until one of the eyes is no longer able to fixate on the object. As a result, this point is the near point of the convergence. Normal near point (loss) of convergence is within the typical range of 6-10 cm for normal eyes but the convergence recovery point (CRP) typically extends to 15 cm. If the NPC is more than 10 cm, there is a sign of poor convergence. Results are noted in terms of NPC, CRP; for example, NPC 7 cm, CRP 12 cm.

When the near point convergence test is performed, as shown in FIG. 4B and FIG. 5B, a pattern of stimuli is shown sequentially on the light bar 20, as in a moving dot, beginning from the middle of the light bar 20 near to the cradle 10 (approximately 20 cm from the subject's eyes), and advancing toward the subject. It should be noted that, although in the view of FIG. 4B, all of the lights 21 on light bar 20 are on, in practice, only one light 21 (i.e. a single dot) is on at a given time. In a particular embodiment, the subject 5 stops the test (for example, by verbal command to the mobile phone 2) when he or she starts to experience double vision. In addition or in the alternative, the light stimuli may be presented as a dot moving gradually closer and closer to the subject (starting roughly from a distance of 20 to moving closer to about 5 cm from subject's eyes). The dot then starts moving away from the subject until reaching back to the original start point. The detection of the points of loss of convergence (an eye drifting outward as the dot approaches the eyes), and, later regain of convergence (as the dot retracts away) are analyzed offline once the data is uploaded to the remote server (on the cloud).

Pupillary Light Reflex (PLR) is defined by systematic constriction of both pupils in response to the onset of a time-limited light stimulus, followed by a refractory dilation period after stimuli offset. The pupil size must change by a non-trivial amount within a specific time frame and should change in both eyes. PLR is a well-established measurement in the management and prognosis of patients with acute brain injuries, in conjunction with other clinical parameters such as age, mode of injury and Glasgow Coma Scale. Typical light stimuli parameters are white (multichromatic) light or blue light (at 465 nm wavelength) with a duration of 1-3 s and typical luminance of 0.001 candelas/square meter (cd/m²). [Ref: Kelbsch et al. (2019) Standards in Pupillography. Frontiers in Neurology. 10: 129.] The PLR test may be administered with the light bar 20 in either configuration, or without a light bar 20 at all.

In the smooth pursuit test (SMP) the patient's ability to accurately track a visual target in a smooth, controlled manner is examined. A user is asked to follow movement of a dot stimulus as it moves along a screen in a smooth, predictable or unpredictable motion. The user is asked to keep his or her eyes directly on the dot without moving the head, and without getting “ahead” or “behind” of the dot. For the performance of this test, it is possible to use a moving dot on the screen of the mobile device 2. The SMP test may be administered with the light bar 20 in either configuration, or without a light bar 20 at all.

While the smooth pursuit test has been executed in a digital manner before, using virtual reality goggles, there are significant benefits in executing the smooth pursuit test in a configuration such as that described herein. First, a doctor or other operator observing the self-administration of the smooth pursuit test may wish to have a clear view of a subject or patient's eyes during performance of the test. When using virtual reality, the patient's eyes are completely hidden; with augmented reality, the patient's eyes may be partially hidden. By contrast, using the system 1, an administrator has a clear view of the patient's eyes throughout performance of the test. Relatedly, an administrator may wish to see clearly the stimuli being presented to the subject's eyes during the smooth pursuit test. This is not easily accessible in virtual reality or augmented reality devices, whereas it is very clearly accessible using system 1. In addition, wearing a heavy assembly such as in standard virtual reality or augmented reality devices, especially following a head injury or concussive event, may be painful or inconvenient for the patient. System 1, by contrast, operates without any equipment on the user's head.

In particular applications, for example, those used in sports, the image sensor 3 of the mobile device 2 may be used to create baseline profiles for particular users. The identity of the user may be manually selected, facially recognized (and matched—e.g., using the image sensor 3), biometrically identified with a fingerprint, or matched through iris recognition. Oculomotor tests may be performed to create and store (either locally or remotely, for example in the cloud) a baseline for a particular subject. Then, for example, when a brain injury (e.g., a concussion is suspected), the same subject can be tested again—recalling the baseline stored event—and comparing the new neurophysiological results with baseline results. Because of its interconnectivity with remote computers, a user may simply use any suitable mobile device (e.g., mobile device 2), log into an account to recall data, and strap-on the add-on instrument to quickly perform test on the side-line of an event.

In another application, for example, oculomotor tests used in detecting drug impairment, the extra features allow testing in diversified environments (e.g., night-time). A police officer may simply be provided the add-on structure 1 with the corresponding application for fitting on an existing mobile device 2 such as a phone or tablet (e.g., iPad). In addition to the oculomotor testing itself, a recordation (e.g., evidence) of the testing can be captured using the video features of the mobile device 2. Both the test results and video evidence can be stored locally or uploaded to the cloud (e.g., remote servers) for later use.

In particular configurations, as referenced herein, such as those used by Drug Recognition Evaluators (DRE), a DRE may desire to manually control the stimuli. Thus, rather than certain tests being automatic, the DRE can manually control the speed and location of the stimuli. Where the light bar 20 is used, this can be accomplished using, for example, a slider on a separate administrative screen for NPC or HGN tests.

In particular configurations, an aggregation of data from multiple testing of a subject or multiple subjects can be cross-referenced to determine patterns associated with different conditions—either to enhance detection of condition or for further research.

In the different types of use cases, the application can provide immediate feedback as necessary to the administrator as a condition (e.g., concussion likely, drug detected, or the like). The feedback may consult cloud-computing (potentially applying predictions of machine-learning models) as necessary if additional processing power is necessary beyond that provided by the mobile device. One of ordinary skill in the art will recognize the benefit-cost analysis between local and cloud computing. As a non-limiting example, cloud computing provides more processing power, but may take longer to access. Regardless of whether cloud computing is used for instantaneous feedback, following a test, the results can later be sent to the remote computers (e.g., the cloud) for storage and/or later analysis. As an example of storage, later testing may reveal that the very particular immediate conditions for a user that yielded a concussion. Such information may be used for training machine learning models for later use on a user-specific basis as an indicator of a concussion.

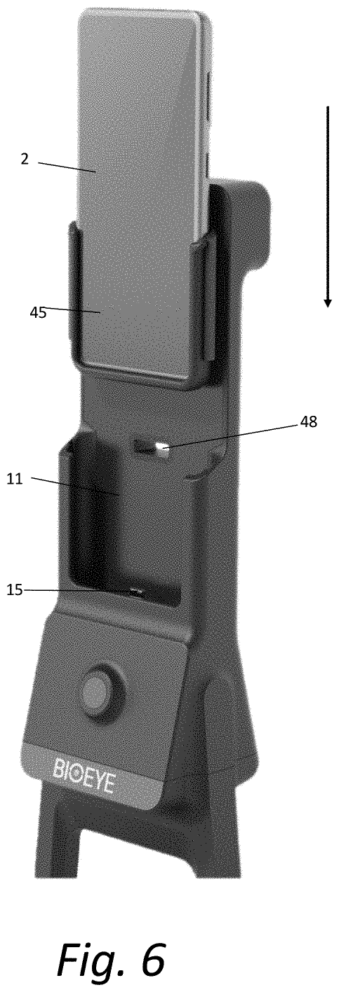

FIGS. 8A and 8B show a second embodiment of an integrated device 50, according to an embodiment of the disclosure. Integrated device 50 is similar in many respects to system 1, and may be used for the same tests. One main difference between integrated device 50 and system 1 is that integrated device 50 is designed to be handheld, whereas system 1 is designed to rest on a flat surface. In addition, integrated device 50 is configured to for administration of tests by a practitioner holding the integrated device 50, whereas system 1 may also be used for self-administration of oculomotor tests by the subject himself or herself, if desired. Other differences in the layout and functioning of the integrated device 50 as compared to the system 1 will become clear through the following description.

Integrated device 50 includes a mobile device 60 and an add-on structure 100. FIG. 8A is a back side of the device 50 that would face a subject being tested whereas FIG. 8B is the front-side of the device 50 that faces an administrator who is running tests.

Mobile device 60 is identical in all relevant respects to mobile device 2. In this particular configuration, mobile device 60 is a smartphone that includes one or more cameras 62. The one or more cameras 62 in this configuration are on the backside 63 of mobile device 60. The backside 63 of the mobile device 60 is also shown.

As with add-on structure 8, add-on structure 100 is generally designed to provide additional functionality that may not exist on the mobile device 60. While described in particular configurations as providing “additional” features, in particular configurations the add-on structure 100 may provide features either that are technically redundant of features of the mobile device 60. This redundancy may either satisfy a universal design to include features that are absent in most mobile devices 60 used or to provide the quality expected for proper operation of the device 50. Further examples will be provided below.

As shown in the configuration of FIGS. 8A and 8B, the add-on structure 100 includes a screen 110, an IR sensor 120, and a clamping portion 118.

At least one infrared illuminator is coupled to screen 110 to illuminate a subject's eyes with infrared light. In the illustrated embodiment, left and right infrared (IR) illuminators 112, 113 are coupled to the screen 110. These IR illuminators may alternatively be placed in different locations on the screen 110.

Also added onto screen 110, or coupled to the screen 110, are stimuli-providing portions 117. While only one is shown, multiple stimulation portions may exist as discrete portions along the screen. The stimuli-providing portions 117 can create a moving dot representation on the screen 110, for example, to which an eye focuses. The stimuli-providing portions 117 are equivalent in all relevant respects to the LED lights 21 previously discussed in connection with the first embodiment.

Also, as either part of the screen 110 or coupled to the screen 110 is a light 115, which may be identical in all relevant respects to light source 12.

The IR sensor 120 captures reflection from the illumination by the left and right infrared IR illuminators 112, 113. IR sensor 120 may be identical in all relevant respects to IR sensor 14. In addition, as discussed above in connection with the first embodiment, an image sensor built in to the mobile device 60, such as camera 62, may be used in place of an IR sensor 120, provided that the built-in image sensor is configured to measure infrared radiation at the desired wavelength. The camera 62 may also be used for recognition of particular individuals who are to be tested, in the manner previously discussed in connection with camera 3.

The clamping portion 118 generally provides an ability to hold portions of the mobile device 60—allowing an interconnection between the add-on structure 100 and the mobile device 60. Any suitable connection mechanisms may be utilized to facilitate the coupling of the mobile device 60. In addition, the clamping portions may facilitate the handling of the integrated device 50. As one non-limiting example, the clamping portions 118 may be coated with material that allows a better grip. While one particular configuration is shown in FIGS. 8A and 8B, other descriptions below provide alternative coupling mechanisms for connecting the mobile device 60 to the add-on structure 100.

While certain components have been described herein, in other configurations, the add-on structure 100 may include more or fewer components. In certain configurations, the components on the add-on structure 100 may depend on the functionality and/or components on the mobile device 60 whereas in other configurations, the components of the add-on structure 100 may not depend on features of the mobile device 60. In the latter configurations, the add-on structure 100 may have features that are redundant to certain features on the mobile device 60. As a non-limiting example, a light 115 may be redundant of a flashlight also on the phone; however, the reason for this perceived redundancy is explained below.

With reference to FIG. 8B, front side 65 of the mobile device 60 may be used for an administrator of the test. In particular configurations, an application or “app” may be loaded on to the mobile device 60 and allow the administrator or subject to interact with the touchscreen features typical of mobile devices 60 in the administration of different tests. The application may be similar in other relevant respects to the application described in connection with the embodiment of FIGS. 1-7.

Optionally, stimuli 117 may have at least a faded portion showing through on a rear side of the screen 110 to allow an administrator to see the location of the stimuli. However, the stimuli 117 need not be of the same appearance or intensity on both sides, as the stimuli 117 serve only as an indicator on the rear side of FIG. 8B.

The length of the screen 110 may be between 40-60 cm for a horizontal reach of 20-30 cm of tracking distance per each side (left and right). Assuming the device 50 is held at 30-40 cm distance from examinee, a 30-45-degree angle (at edge) would form for the horizontal gaze nystagmus (HGN) testing. The width of screen 110 may be approximately 1 cm, and the dot diameter may be approximately 0.5 cm, as described in connection with light bar 20. The thickness of the add-on structure 100 may be approximately 0.5 cm—providing robustness and preventing the add-on structure 100 from bending or breaking.

In particular configurations, portions of the add-on structure 100 may be foldable allowing for convenient carrying. In addition, for purpose of the near point convergence (NPC) test, a segment of the screen 110 may have the ability to tilt perpendicularly (facing the examinee), facilitating a nearing or retracting dot scenario (as shown in FIG. 8).

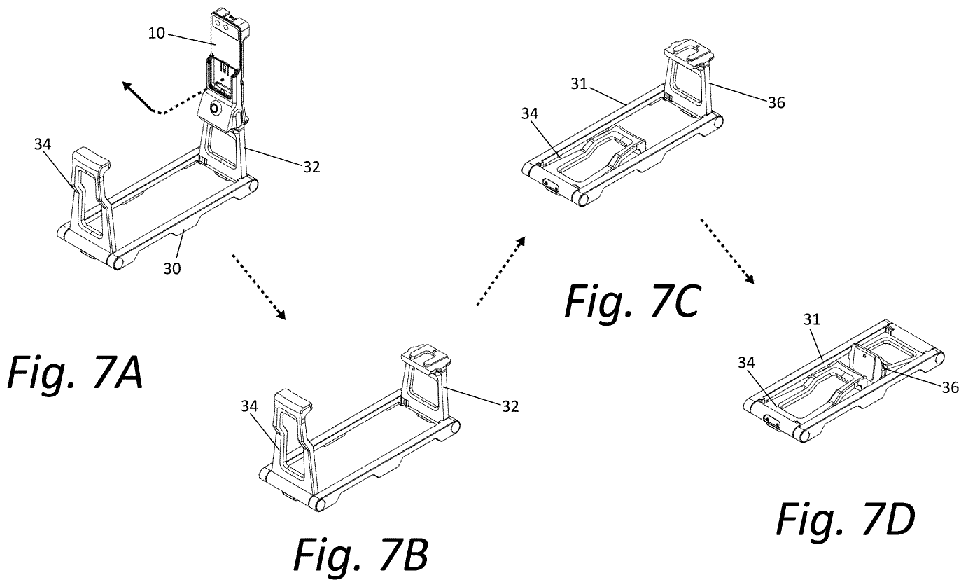

FIG. 9 shows the location of the stimuli 117 and how the screen 110 can be moved toward a subject for a nearing or retracting dot scenario test. In a similar fashion to movement of the screen 110, in particular configurations, the screen 110 may be folded for storage. Such folding may occur in any suitable manner.

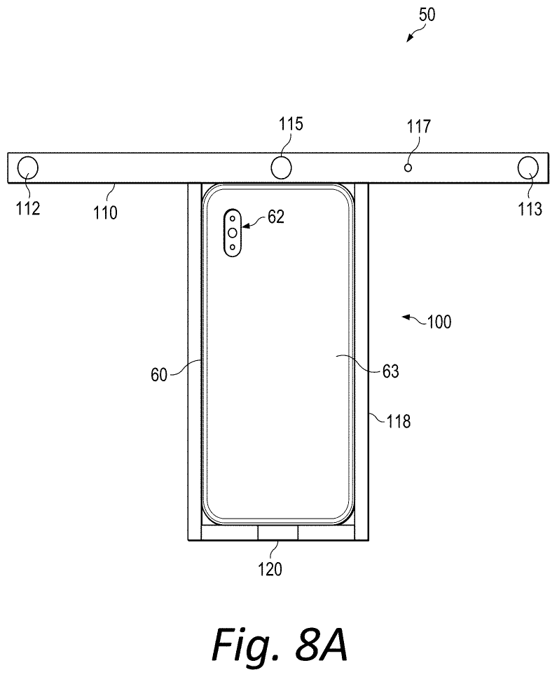

FIG. 10 shows an example use of the device of FIGS. 8A, 8B, and 9 with a subject 200, according to an embodiment of the structure, in a position for performing one or more of the above-described tests.

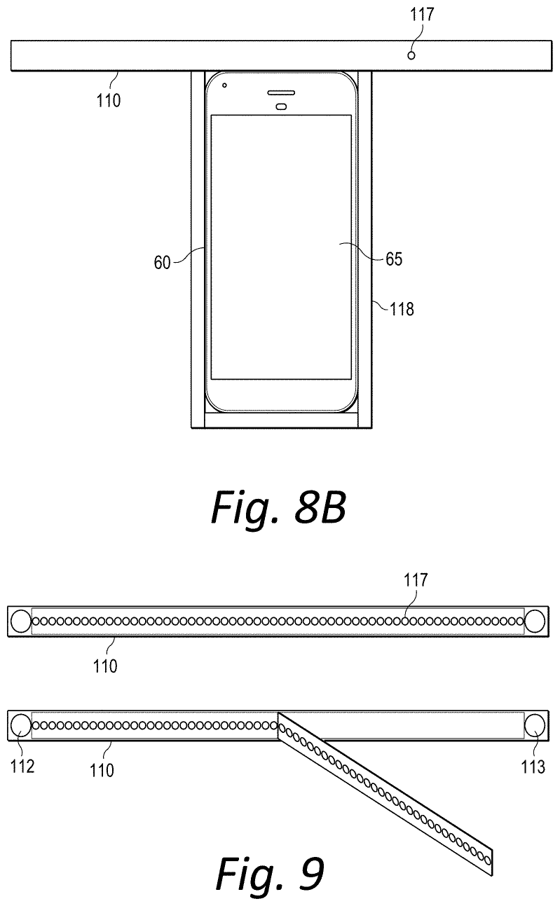

FIGS. 11A, 11B, and 11C illustrate varying holders and corresponding features that may be utilized, according to embodiments of the disclosure. While such holders are shown, it should be understood that other holders may additionally be utilized and avail from embodiments of the disclosure.

With reference to FIG. 11A, the mobile device 60A is shown as being tilted. As part of the add-on structure 100A, a holder or handle is provided to an administrator to facilitate stable posture and convenient administration of tests.

With reference to FIG. 11B, the mobile device 60B is mounted on an add-on structure 100B that includes a gimballed device—to keep the mobile device steady, even through small movements. The gimballed-device may support both vertical and horizontal positioning of the mobile device 60B. In particular configurations, the gimbals may be mechanically operated to keep such stability. In addition, according to certain configurations, the test subject can be selected as an active tracking object—with communication feedback to mechanical gimbals to make sure the object is kept in focus. Such a selection of features is shown with reference to FIG. 11C on the front side screen 65. In particular, a face of the subject 200 is selected (as indicated by the bracket 65B surrounding the face) and mechanic gimbals keep the bracketed face in focus—even through slight movements of the mobile device. An example mechanical gimballed device that receives a mobile device and has active tracking features is sold by DJI of Shenzhen, China under the name OSMO MOBILE. Yet other gimbaled devices or mechanical stabilizers may alternatively be utilized.

Feedback for the gimballed device can be obtained from the one or more cameras of the mobile device, the IR sensor, another sensor, or a combination of the proceeding.

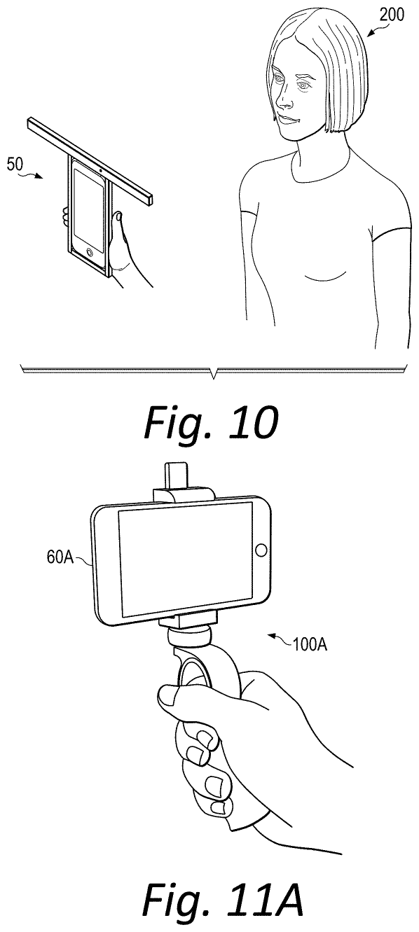

FIGS. 12-15 address various specialized aspects of using system 1 or integrated device 50 for performance of oculomotor tests, according embodiments of the present disclosure.

FIG. 12 illustrates how one may triangulate an image distance to a subject 200 using at least two image sensors 191, 192. In particular configurations, the integral camera from the mobile device 3 or 62 and the IR sensor 14 or 120 (residing at a known distance from one another) may be used to determine the distance of the subject. In the embodiment of FIGS. 8A-8B, when the mobile device 60 includes multiple cameras, the integral functions of such mobile devices which use their sensors for distances may be utilized. As non-limiting examples, newer applications on such mobile devices have measuring features to measure, for example, the distance between objects—using built-in sensors. Such features of the phone (where they exist on the mobile device 2) can also be used.

In alternative configurations, Time-of-Flight (ToF) cameras may be utilized—either obtained from mobile device built-in-features or using one from the add-on structure. ToF camera sensor manufacturers/devices include, but are not limited to AMS/Heptagon, TeraRanger One, ASC TigerCub, Riegl, and Lucid/Helios.

Like the sensing of the distance to the test subject 200, certain configurations may use the same sensor (combination of sensors in the mobile device and newly added ones with the add-on structure) to similarly measure the distance of a stimuli pen (or stimulate light pen) from the device for an understanding of the relative angle of the eye being measured. In such configurations, a light bar 20 or screen 110 need not be utilized.

Other sensors that may be utilized in certain configurations are one or more 360-degree cameras that use fish eye effects to capture a large area. Such cameras may serve a dual purpose in some configurations of capturing both a stimuli pen and the subject—for distance determination. Exemplary small 360-degree cameras are sold by Insta360 of Shenzhen, China under the name INSTA360.

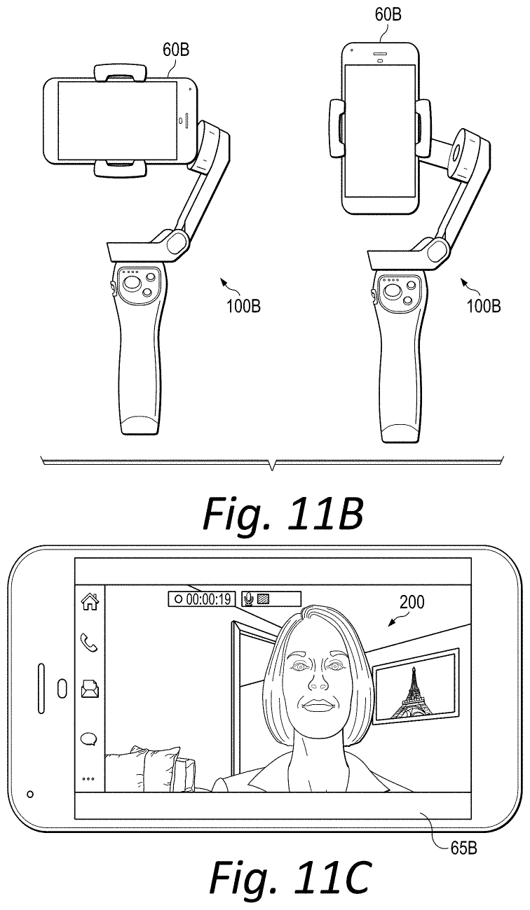

FIGS. 13A and 13B illustrate additional measurement techniques, according to embodiments of the disclosure. FIGS. 13A and 13B show the use of propagated electromagnetic waves to, for example, triangulate and detect both the distance of and relative angle between an object (e.g., stimuli pen 198) and the test subject (e.g., using an emitter 199 that may be placed on a nose of the test subject 200).

While one antenna can determine distance, two or three (or even four) can better approximate. Three antennas are shown in FIGS. 13A and 13B. The antennas themselves can either be completely in the add-on structure 1 or 100 or at least some can be on the antennas of the mobile device 2 or 60. One of ordinary skill in the art will recognize how the same signal propagated and received by three separate antennas can be used to determine distance and direction. Such techniques are commonly used in a much larger scale for GPS and Cell-tower triangulation. Here, the same techniques are used for much smaller scale triangulation to achieve millimeter level accuracy.

With a configuration such as that shown, all three items (device, subject, and stimuli) are known over time in a three-dimensional space relative to one another and can be correlated with the eye movement.

In particular configurations, different technologies described herein may be combined.

FIG. 14 is a simplified block diagram illustrative of a communication system 700 that can be utilized to facilitate communication described herein. Each item associated with a communication is generically described as endpoint 710, 720 which communicates through a path or communication network 730. With reference to the FIGURES described above, one endpoint may be the mobile device whereas the other endpoint may be the add-on structure. Likewise, one endpoint may be the mobile device whereas the other endpoint may be a cloud-computing processor, storage, or both.

When referencing communication, for example, showing arrows or “clouds,” or “networks,” any of such communication may occur in the manner described below or other manners. Or, a communication can be local, for example, over USB-C or Bluetooth. Likewise, the endpoints may generally correspond to any two particular components described (or combination of component) with another component or combination of components.

As used herein, “endpoint” may generally refer to any object, device, software, or any combination of the preceding that is generally operable to communicate with and/or send information to another endpoint. In certain configurations, the endpoint(s) may represent a user, which in turn may refer to a user profile representing a person. The user profile may comprise, for example, a string of characters, a user name, a passcode, other user information, or any combination of the preceding. Additionally, the endpoint(s) may represent a device that comprises any hardware, software, firmware, or combination thereof operable to communicate through the communication path or network 730.

Examples of an endpoint(s) include, but are not necessarily limited to those devices described herein, a computer or computers (including servers, applications servers, enterprise servers, desktop computers, laptops, netbooks, tablet computers (e.g., IPAD), a switch, mobile phones (e.g., including IPHONE and Android-based phones), networked televisions, networked watches, networked glasses, networked disc players, components in a cloud-computing network, or any other device or component of such device suitable for communicating information to and from the communication path or network 730. Endpoints may support Internet Protocol (IP) or other suitable communication protocols. In particular configurations, endpoints may additionally include a medium access control (MAC) and a physical layer (PHY) interface that conforms to IEEE 802.11. If the endpoint is a device, the device may have a device identifier such as the MAC address and may have a device profile that describes the device. In certain configurations, where the endpoint represents a device, such device may have a variety of applications or “apps” that can selectively communicate with certain other endpoints upon being activated.

The communication path or network 730 and links 715, 725 to the communication path or network 730 may include, but is not limited to, a public or private data network; a local area network (LAN); a metropolitan area network (MAN); a wide area network (WAN); a wireline or wireless network (WIFI, GSM, CDMA, LTE, WIMAX, BLUETOOTH or the like); a local, regional, or global communication network; portions of a cloud-computing network; a communication bus for components in a system; an optical network; a satellite network; an enterprise intranet; other suitable communication links; or any combination of the preceding. Yet additional methods of communications will become apparent to one of ordinary skill in the art after having read this specification. In particular configuration, information communicated between one endpoint and another may be communicated through a heterogeneous path using different types of communications. Additionally, certain information may travel from one endpoint to one or more intermediate endpoint before being relayed to a final endpoint. During such routing, select portions of the information may not be further routed. Additionally, an intermediate endpoint may add additional information.

Although an endpoint generally appears as being in a single location, the endpoint(s) may be geographically dispersed, for example, in cloud computing scenarios. In such cloud computing scenarios, an endpoint may shift hardware during back up. As used in this document, “each” may refer to each member of a set or each member of a subset of a set.

When the endpoints(s) 710, 730 communicate with one another, any of a variety of security schemes scheme may be utilized. As an example, in particular embodiments, endpoint(s) 720 may represent a client and endpoint(s) 730 may represent a server in client-server architecture. The server and/or servers may host a website. And, the website may have a registration process whereby the user establishes a username and password to authenticate or log in to the website. The website may additionally utilize a web application for any particular application or feature that may need to be served up to web site for use by the user.

A variety of embodiments disclosed herein may avail from the above-referenced communication system or other communication systems.

FIG. 15 is an embodiment of a general-purpose computer 810 that may be used in connection with other embodiments of the disclosure to carry out any of the above-referenced functions and/or serve as a computing device for endpoint(s) 710 and endpoint(s) 720.

General purpose computer 810 may generally be adapted to execute any of the known OS2, UNIX, Mac-OS, Linux, Android and/or Windows Operating Systems or other operating systems. The general-purpose computer 810 in this embodiment includes a processor 812, random access memory (RAM) 814, a read only memory (ROM) 816, input device 818, one more camera(s) 824, input devices 820, sensors 822, a display 826 and a communications link 928. In other embodiments, the general-purpose computer 810 may include more, less, or other component parts. Embodiments of the present disclosure may include programs that may be stored in the RAM 814, the ROM 816 or other storage devices and may be executed by the processor 812 in order to carry out functions described herein. The communications link 828 may be connected to a computer network or a variety of other communicative platforms including, but not limited to, a public or private data network; a local area network (LAN); a metropolitan area network (MAN); a wide area network (WAN); a wireline or wireless network; a local, regional, or global communication network; an optical network; a satellite network; an enterprise intranet; other suitable communication links; or any combination of the preceding. In certain configurations, antennas may be utilized.

The camera may include a variety of types of cameras—capturing either images or infrared reflections. The sensor may include any suitable sensor for capturing environmental parameters or other items for processing and feedback.

The input device 820 may include a tactical input device associated with the display 826, a keyboard, a mouse, or other input device.

Although FIG. 15 provides one embodiment of a computer that may be utilized with other embodiments of the disclosure, such other embodiments may additionally utilize computers other than general purpose computers as well as general purpose computers without conventional operating systems. Additionally, embodiments of the disclosure may also employ multiple general-purpose computers 810 or other computers networked together in a computer network. The computers 810 may be servers or other types of computing devices. Most commonly, multiple general-purpose computers 810 or other computers may be networked through the Internet and/or in a client server network. Embodiments of the disclosure may also be used with a combination of separate computer networks each linked together by a private or a public network.

Several embodiments of the disclosure may include logic contained within a medium. In the embodiment of FIG. 15, the logic includes computer software executable on the general-purpose computer 810. The medium may include the RAM 814, the ROM 816, or other storage structure. In other embodiments, the logic may be contained within hardware configuration or a combination of software and hardware configurations.

The logic may also be embedded within any other suitable medium without departing from the scope of the disclosure.

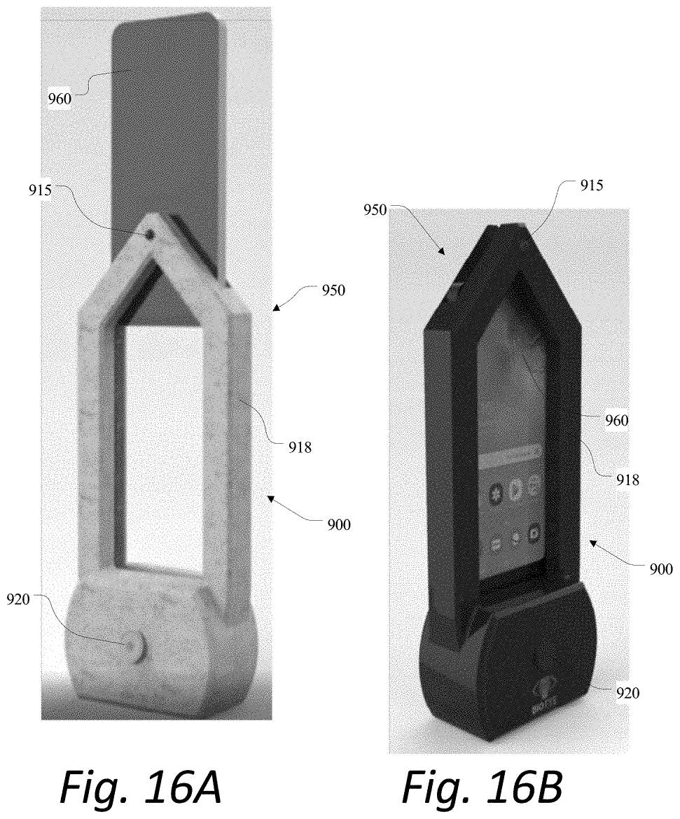

FIGS. 16A and 16B show a third configuration of an integrated device 950, according to an embodiment of the disclosure. The integrated device 950 may include features similar to those described above with reference to the previous embodiments—including an add-on structure 900, a mobile device 960, a clamping portion 918, an IR sensor 920, and a light 915. The integrated device 900 may operate in a similar manner to the integrated devices previously described—including use of an application loaded on the mobile device.

Additionally, operationally, the integrated devices 950 may operate in accordance with any of the configurations described herein. With a specific reference to FIGS. 12, 13A, and 13B, the referenced sensor and/or antennas may be incorporated in any portion of the add-on structure 900. Similarly, a stimuli pen 198 may be used. Or, a screen 110 may be used. Yet, alternatively, other stimuli objects in communication with either the mobile device 960 or add-on structure 900 may be used. Suitable communications include, but are not limited to, Bluetooth and Wi-Fi. Others are described with reference herein that may also be used.

It will be understood that well known processes have not been described in detail and have been omitted for brevity. Although specific steps, structures and materials may have been described, the present disclosure may not be limited to these specifics, and others may substitute as is well understood by those skilled in the art, and various steps may not necessarily be performed in the sequences shown.

While this disclosure has described certain embodiments and generally associated methods, alterations and permutations of these embodiments and methods will be apparent to those skilled in the art. Accordingly, the above description of example embodiments does not define or constrain this disclosure. Other changes, substitutions, and alterations are also possible without departing from the spirit and scope of this disclosure, as defined by the following claims.

What is claimed is:

1. A system for the administration of oculomotor tests with a mobile device, comprising:
a cradle having a cavity configured to receive the mobile device therein;
at least one light and at least one light sensor arranged on the cradle;
an elongated light bar attachable to and electrically connectable to the cradle;
and a stand comprising:
an elongated frame configured to rest flat on a horizontal surface, the elongated frame comprising a first end, a second end, and a long axis defined between the first and second ends;
a chin rest arranged at the first end;
and a cradle support arranged at the second end and supporting the cradle thereon.

2. The system of claim 1, wherein:
the chin rest is foldable between an upright position and a folded position, wherein in the upright position the chin rest is perpendicular to the long axis, and in the folded position the chin rest is parallel to the long axis; and
the cradle support is foldable between an upright position and a folded position, wherein in the upright position the cradle support is perpendicular to the long axis, and in the folded position the cradle support is parallel to the long axis; wherein, when the chin rest and cradle support are both in the upright position, the chin rest and cradle support are parallel to each other.

3. The system of claim 1, wherein the length of the long axis is approximately 40 cm.

4. The system of claim 1, wherein the cradle support and cradle are removably attached to each other.

5. The system of claim 1, further comprising an adapter formed of a thermoplastic polymer, wherein said adapter is configured to be arranged within the cavity between the mobile device and the cradle to thereby stabilize the mobile device.

6. The system of claim 1, wherein the at least one light comprises a white light source configured to provide stimuli for relevant oculomotor tests and an infrared light source arranged above the cavity and configured to provide infrared light for purposes of improving facial IR image sensing.

7. The system of claim 1, wherein the at least one light sensor comprises an infrared sensor arranged below the cavity.

8. The system of claim 1, wherein the light bar is attachable to the cradle in a first orientation in which the light bar is attached to the cradle at a centerpoint of the light bar, and is centered over the cradle, and a second orientation in which the light bar is attached to the cradle at an edge of the light bar, and overhangs over the elongated frame.

9. The system of claim 1, further comprising a connector arranged within the cradle for forming a data connection between the cradle and the mobile device.

10. The system of claim 1, further comprising an application stored in a non-transitory memory of the mobile device, said application configured to coordinate administration of one or more oculomotor tests using one or more of the at least one light, at least one light sensor, and the light bar.

11. The system of claim 1, wherein the application is further configured to coordinate administration of the one or more oculomotor tests using a built-in sensor of the mobile device.

12. The system of claim 1, wherein the application includes instructions for self-administration of the one or more oculomotor tests for a subject whose chin is resting on the chin rest.

13. A method comprising:
inserting a mobile device into a cavity of a cradle, the cradle being attached to a cradle support of a stand, wherein the stand comprises an elongated frame configured to rest flat on a horizontal surface, the elongated frame comprising a first end, a second end, and a long axis defined between the first and second ends; a chin rest arranged at the first end; and the cradle support arranged at the second end, and the cradle comprises the cavity configured to receive a mobile device therein, at least one light, and at least one light sensor arranged on the cradle;
forming a data connection between the mobile device and the cradle; and
operating an application stored in a non-transitory memory of the mobile device, thereby coordinating administration of one or more oculomotor tests, on a subject whose chin is resting on the chin rest, using one or more of the at least one light and the at least one light sensor.

14. The method of claim 13, wherein the one or more oculomotor tests comprises a pupillary light reflex test or a smooth pursuit test.

15. The method of claim 13, further comprising attaching a light bar to the cradle, and wherein the operating step comprises coordinating administration of the one or more oculomotor tests using the light bar.

16. The method of claim 13, wherein the operating step comprises coordinating administration of the one or more oculomotor tests using a built-in sensor of the mobile device.

17. The method of claim 13, wherein the operating step comprises self-administering the one or more oculomotor tests.

18. A system for the administration of oculomotor tests with a mobile device, comprising:
a cradle having a cavity configured to receive the mobile device therein and an infrared light source configured to provide infrared light for purposes of improving facial infrared image sensing;
a connector arranged within the cradle for forming a power connection between the cradle and the mobile device, serving to power the infrared light source; and
a filter arranged on an image sensor of the mobile device, the filter configured to pass infrared light and to block visible light.

19. The system of claim 18, wherein the connector further provides a data connection between the cradle and the mobile device, and the cradle further comprises a white light source configured to provide stimuli for relevant oculomotor tests.

20. The system of claim 18, wherein the connector further provides a data connection between the cradle and the mobile device, and further comprising an elongated light bar attachable to and electrically connectable to the cradle.
